(19) United States
(12) Patent Application Publication
(10) Pub. No.: US 2022/0152216 A1
(43) Pub. Date: May 19, 2022

(54) ANTIBODY-POLYMER CONJUGATES

(71) Applicant: BOGAZICI UNIVERSITESI, Istanbul (TR)

(72) Inventor: Rana Sanyal, Istanbul (TR)

(21) Appl. No.: 17/587,667

(22) Filed: Jan. 28, 2022

Related U.S. Application Data
(63) Continuation of application No. PCT/TR2020/050674, filed on Jul. 29, 2020.

(30) Foreign Application Priority Data
Jul. 30, 2019 (TR) 2019/11542

(51) Int. Cl. A61K 47/68; A61K 45/06; A61K 47/58

(52) U.S. Cl. CPC A61K 47/6883; A61K 47/6803; A61K 47/58; A61K 47/6855; A61K 45/06

(57) ABSTRACT

The present invention relates to antibody-polymer conjugates which have high drug carrying capacity and which can realize active targeting thanks to the antibody existing thereon.

### CROSS REFERENCE TO RELATED APPLICATIONS

This application is a continuation of PCT International Application No. PCT/TR2020/050674, filed Jul. 29, 2020, which claims priority to Turkish Application No. 2019/11542, filed Jul. 30, 2019, which are hereby incorporated by reference in its entirety.

### TECHNICAL FIELD

The present invention relates to antibody-polymer conjugates which have high drug carrying capacity and which can realize active targeting thanks to the antibody existing thereon.

### PRIOR ART

The conjugation of humanized or non-humanized monoclonal antibodies by means of chemical bond with cytotoxic small molecule drugs has led to important changes in the design of cancer chemotherapy. (Chari et al, 2014; Perez et al, 2014; Bouchard et al, 2014; Jain et al, 2015; McCombs and Owen, 2015; Chudasama et al., 2016; Diamantis and Banerji, 2016).

The most important advantage provided by antibody drug conjugates is that they provide treatment which is unique to the target and that low cytotoxicity is observed in healthy cells except the target and that low immunogenicity is observed.

In preparation of antibody-drug conjugates, cleavablecleavable bonds like hydrazone, disulphide can be used, or cleavablenon-cleavable bonds like amide can be used.

In the known art, there are antibody-drug conjugates which are sold in the market. As an example to these, Brentuximab vedotin (Adcetris®) can be shown. Adcetris comprises Brentuximab (Mab) which is a monoclonal antibody and mono-methyl auristatin as drug. Said monoclonal antibody and drug molecule are bound to each other by means of Val-Cit linker which can be cleaved in the body by means of Cathepsin B enzyme. In such conjugates, there are on the average 4 drug molecules per an antibody and these are connected to one other by means of a linker cleavable in the body. The bond which exists in the conjugate which reaches the target tumor cell with the help of monoclonal antibody is cleaved here by means of cathepsin B enzyme, and mono-methyl auristatin which is a cyto-toxic agent is released to the tumor region. This way, mono-methyl auristatin molecule, which is not possible to be used in any manner due to high toxicity, can be used for treatment.

Another antibody-drug conjugate sold in the market is Trastuzumab emtansine known with the trade name Kadcyla®. In this product, trastuzumab is used as the antibody, and cytotoxic emtansine, having abbreviation DM1, is used as drug. While trastuzumab binds to the HER2 receptor in a stand-alone manner and stops growth of cancer cells, trastuzumab-emtansine conjugate is carried into the cell by means of receptor and it is catabolized by lysosomes, and by means of this, emtansine is released. The released emtansine is bound to tubulin and stops mitotic cycle and thus, it leads to death of the cancer cell. Since trastuzumab is specific to the HER2 receptor and since HER2 receptor is expressed more than normal only in cancer cells, trastuzumab-emtansine conjugate is specifically transferred to the tumor cells.

In this conjugate, the bond between the drug and the antibody is provided by means of succinimidil trans-4-(maleimidil-methyl)cyclo-hexane-1-carboxylate (SMCC). While the thiol group, which exists on emtansine, reacts with maleimide, the succinimide group which exists on SMCC reacts with lysine which exists on trastuzumab, and amide bond is formed. Approximately 3.5 drug molecules exist for each trastuzumab.

Kopecek et. al., 2018 (Eur. J. Pharm. Sci., 2017 103, 36-46) has developed Epirubicin which is copolymerized with conjugated HPMA by means of Rituximab in order to increase treatment efficiency and patient tolerance in B-cell lymphoma patients. Since the water solubility of the HPMA monomer used in obtaining these conjugates is delimited, a disadvantage occurs.

The antibody-drug conjugates, which are present in the known state of the art, are obtained by means of binding the drug to the antibody by means of a cleavable or non-cleavable bond as in the Adcetris® and Kadcyla® examples given above. In such conjugations, the drug-antibody proportion is in general approximately 4 on the average. In other words, on an antibody, there are drug molecules between 0 and 8, on average there are 4 drug molecules. For the efficiency of the treatment, it is preferred that there are more drug molecules on the antibody, however, when the antibody is modified for providing more drug molecules, the activity of monoclonal antibody decreases, in other words, binding ability decreases.

Moreover, since small amount of drug can be carried in such antibody-drug conjugates, drugs are used which are toxic such that they cannot be given normally in a stand-alone manner. When even a small part of these drugs breaks from the antibody-drug conjugate without reaching the tumor, these drugs lead to serious side effects.

Another problem of the known antibody-drug conjugates occurs during chemical modification of the antibody for binding the linkers or drugs. In order to form an area which is suitable for reaction on the antibody, the disulphide bonds which exist in the antibody structure are broken by means of chemicals named as DTT or TCEP, and the thiol reactive groups are obtained. Since this process leads to a change in the 3-dimensional structure in said antibody, it leads to a decrease in the bonding ability to the related receptor.

The inventors prepared the subject matter antibody-polymer conjugates for increasing the drug amount per antibody and at the same time for developing antibody-polymer conjugates which have higher solubility when compared with the antibody-polymer conjugates which are present in the known art.

### BRIEF DESCRIPTION OF THE INVENTION

The inventors have prepared a drug carrying system (antibody-polymer conjugate) formed by polymer which carries therapeutic agent that has been conjugated with cleavable or non-cleavable bonds to monoclonal antibody and said polymer is characterized by comprising (meth)acrylate backbone and poly(ethylene glycol) at the side branches and the therapeutic agent is connected to the polymer backbone by means of a cleavable linker.

Thanks to the antibody-polymer conjugates according to the invention, the amount of drug per polymer can be changed as desired. Moreover, since the polymers which exist in the antibody-polymer conjugates according to the invention carry more than one drug molecule in each chain, when said polymers are conjugated to an antibody, the drug:antibody proportion is also increased. Thus, the drugs with less cytotoxic effect are directly sent to the tumor tissue by means of the conjugate. Moreover, the poly(ethylene glycol) chains which exist in side branches of the polymer structure provide obtaining antibody-polymer conjugates with high water solubility and thus high bioavailability.

### DETAILED DESCRIPTION OF THE INVENTION

In a preferred embodiment of the present invention, the antibody-polymer conjugates are shown by Formula 1;

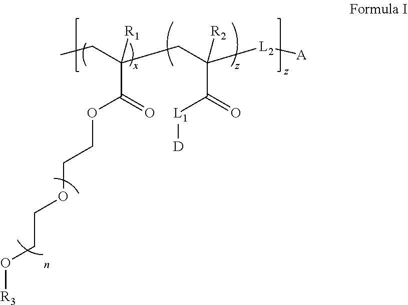

wherein;

R₁, R₂, R₃ are independently selected from —H or —CH₃

x is a natural number between 1 and 140

y is a natural number between 1 and 40

z is a natural number between 1 and 15

L₁ is a cleavable linker

L₂ is a linker

D is a therapeutic agent

A is a monoclonal antibody.

The term “antibody-polymer conjugate” used within the scope of the present invention describes the polymer structure connected in a covalent manner to an antibody. The polymeric structures mentioned within the scope of the present invention are polymers having (meth)acrylate backbone and comprising therapeutic agent and poly(ethylene glycol) bound to the polymer backbone by means of covalent bonds at the side chains.

The terms of “antibody-polymer conjugate”, “formula 1”, “antibody-polymer conjugate shown by formula 1” that are used within the scope of the present invention are equivalent to one other and can be used interchangeably.

The term “(meth)acrylate” used within the scope of the present invention indicates “acrylate” and “meth-acrylate” terms. Therefore, “(meth)acrylate” can be used instead of “acrylate” and “methacrylate” and comprises all of the above mentioned characteristics of these terms.

The term “cleavable linker” used within the scope of the present invention describes the bonds which connect the polymer backbone and the therapeutic agent to each other and which can be broken in the presence of a specific stimulant. Here, the stimulant can be any item which can provide breaking of said bond, for instance, light, chemical agent, biologic agent, any substance which can exist in the body of a mammalian, heat etc.

The term “linker” used within the scope of the present invention describes the bonds which connect the polymer backbone and the antibody to each other. Here, the linkers can be cleavable or non-cleavable.

The term “therapeutic agent” used within the scope of the present invention describes all agents used for eliminating, for decelerating, for preventing any physiological condition, for decreasing symptoms or for providing elimination of the symptoms or for detecting the presence of a physiological condition. Said agents can have small molecules, macro-molecules or a biological-based structure or a structure which mimics biological based molecules.

The term “monoclonal antibody” used within the scope of the present invention describes the antibodies which show reaction against only one epitope and which are based on only one B-lymphocyte. The terms “monoclonal antibody” and “antibody” used within the scope of the present invention are equivalent to one other and can be used interchangeably.

The number shown with z provided in the scope of the present invention describes the number of polymer chains connected to an antibody. In a preferred embodiment of the present invention, z describes a number between 1.05 and 14 and preferably a number between 1.5 and 13. In an embodiment of the present invention, z can be a number between 2.0 and 12 or between 2.5 and 11 or between 3 and 10. For instance, z can be a number like 1.05, 1.1, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0.

The drug:antibody ratio which exists in the antibody polymer conjugates of the invention, in other words, the amount of drug molecule per antibody, can be between 1:1 and 100:1. In other words, in the subject matter antibody-polymer conjugates, a drug between 1 and 100 molecules can be provided per antibody. In a preferred embodiment of the present invention, the drug antibody ratio can be between 4:1 and 90:1, and in a particularly embodiment, it can be between 6:1 and 80:1.

The term “poly(ethylene glycol)” used within the scope of the present invention indicates a polyether compound which has the structure of H—(O—CH₂—CH₂)n-OR₃ and n is a natural number between 1 and 200 and R₃ is selected from H or —CH₃. PEG is defined as an oligomer or polymer of the ethylene oxide. The terms of “PEG”, “polyethylene glycol”, “polyethylene oxide”, “PEO”, “poly-oxy-ethylene” and “POE” indicate the same structure and can be interchangeably used in this specification.

The polymer which has poly(ethylene glycol) and therapeutic agent in the subject matter side chains can be a random copolymer or block copolymer structure. The term “random copolymer” describes a copolymer where the monomers forming the copolymer do not follow any specific sequence. The term “block copolymer” describes a copolymer where all of a kind of monomer are grouped together and where the other type of monomer are grouped together. In a preferred embodiment of the present invention, the polymer having poly(ethylene glycol) and therapeutic agent is in the structure of random copolymer.

In a preferred embodiment of the present invention, in the subject matter antibody-polymer conjugates, the therapeutic agent can be selected from a group comprising anti-neoplastic agents, immune system modulating agents, agents which show anti-angiogenic characteristic on malign cells, agents which inhibit cell proliferation on malign cells or agents which increase immune cell proliferation.

The term “malign cells” used here describes the cells where the cell cycle is rapid and/or where the genetic structure has changed and/or which grow in an uncontrolled manner and/or which have increased mobility and/or where the cell surface structure has changed and/or where lytic factor secretion has changed and/or which show invasive growth and/or which have chemotaxis characteristic and/or which have abnormal shape and/or size and/or where cyto-plasma has decreased and/or which have big core and/or which have grown nucleus pores.

The term “immune cell” used herein describes all of the cells which exist in a mammalian body, preferably the cells which exist in human body. Said cells can show activity in different manners in the immune system. The immune cells described within the scope of the present invention can be B-cells, dendritic cells, granulocytes, natural lymphoid cells, mega-karyocytes, monocytes, macrophages, myeloid-based suppressive cells, platelets, red blood cells, T-cells and thymocytes.

Anti-neoplastic agent can be selected from a sub-group comprising but not limited to nucleoside analogs, antifolates, other metabolites, topoisomerase I inhibitors, anthracyclins, podophyllotoxins, taxanes, vinca alkaloids, alkylating substances, platinum compounds, anti-hormones, radio-pharmaceuticals, tyrosine kinase inhibitors, rapamycin protein complex mammalian target (mTOR) inhibitors, retinoids, immune system regulating substances, histone deacetyase inhibitors and other substances.

Nucleoside analogs can be selected from a group comprising but not limited to azacitidin, cladribin, chlopharabin, citarabin, decitabin, floxuridin, fludarabin, florouracil (5-FU), gemcitabine, mercaptopurine, nelarabin, pentostatin, tioguanin, trifluoridin and tipiracil.

Antifolates can be selected from a group comprising but not limited to metotreksat, pemetrexed, pralatrexed and raltitrexed.

The other metabolites can be selected from a group comprising but not limited to hydroxy-carbamide.

Topoisomerase I inhibitors can be selected from a group comprising but not limited to irinotecan and topotecan.

Anthracyclins can be selected from a group comprising but not limited to daunorubicin, doxorubicin, epirubicin, idarubicin, mitoxantron and valrubicin.

Podophyllotoxins can be selected from a group comprising but not limited to etopocid and tenipocid.

Taxanes can be selected from a group comprising but not limited to cabazitaxel, docetaxel and paclitaxel.

Vinca alkaloids can be selected from a group comprising but not limited to vinblastin, vincristin, vindesin, vinflunin and vinorelbin.

Alkylating substances can be selected from a group comprising but not limited to bendamustin, chlorambucil, dacarbazine, melphalane, streptozotosin and trabestedin.

Antihormon compounds can be selected from a group comprising but not limited to abirateron, bicalutamid, siproteron, degarelix, exemestane, fulvestrant, goserelin, histrelin, leuprolide, miphepristone and triptorelin.

Tyrosine kinase inhibitors can be selected from a group comprising but not limited to afatinib, aksitinib, bosutinib, kobimetinib, crizotinib, dasatinib, erlotinib, gefitinib, imatinib, lapatinib, nilotinib, osimertinib, pazopanib, ruksolitinib, sunitinib and vandetanib.

Rapamycin protein complex mammalian target (mTOR) inhibitors can be selected from a group comprising but not limited to everolimus and temsirolimus.

Retinoids can be selected from a group comprising but not limited to alitretinoin, bexarotene, isotretinoin, tamibarotene and tretinoin.

Immune system regulation substances can be selected from a group comprising but not limited to lenalidomid, pomalidomid and talidomid.

Histone deacetylase inhibitors can be selected from a group comprising but not limited to belinostat, panobinostat, valproate and vorinostat.

The other substances can be selected from a group comprising but not limited to anagrelide, seritinib, dabrafenib, idelalisib, ibrutinib, palbocyclib, vemuraphenib, bleomisin, bortezomib, dactinomisin, eribulin, estramustine, ixabepilone, mitomisin, prokarbazin, alectinib, fluxymesteron, iobenguane, imiguimod, interferon, iksazomib, Ianreotid, lentinan, octreotid, omacetaxine, tegafur, gimerazil, oterasil, uracil, kombretastatin, emtansine and chloroquine.

In a preferred embodiment of the present invention, the therapeutic agent can be selected from antineoplastic agents, preferably from taxanes, antifolates, tyrosine kinase inhibitors, anthracyclins, nucleoside analogs or other substances.

In a most preferred embodiment, the therapeutic agent is selected from a group comprising docetaxel, pemetrexed, hydroxchloroquine, combretastatin, gemcitabine, doxorubicin, 5-Fluorouracil (5-FU), 5′-Deoxy 5-Fluorocytidine (5′-DFCR), emtansine and lapatinib.

In an embodiment of the present invention, the therapeutic agent is docetaxel. In an embodiment of the present invention, the therapeutic agent is hydroxychloroquine. In an embodiment of the present invention, the therapeutic agent is combretastatine. In an embodiment of the present invention, the therapeutic agent is gemcitabine. In an embodiment of the present invention, the therapeutic agent is doxorubicin. In an embodiment of the present invention, the therapeutic agent is 5-FU. In an embodiment of the present invention, the therapeutic agent is 5′-deoxy 5-fluorocytidine (5′-DFCR). In an embodiment of the present invention, the therapeutic agent is lapatinib. In an embodiment of the present invention, the therapeutic agent is emtansine.

In an embodiment of the present invention, the therapeutic agent is a combination of two or more therapeutic agents selected from the group comprising docetaxel, pemetrexed, chloroquine, combretastatine, gemcitabine, doxorubicin, 5-FU, 5′-Deoxy 5-Fluorocytidine (5′-DFCR), emtansine and lapatinib.

The therapeutic agent can be present at an amount between 1% and 40% of the polymer weight provided on the antibody-polymer conjugate, preferably at an amount between 2% and 35% and particularly preferably between 3% and 30%. The therapeutic agent can be present at an amount between 4% and 25% or between 5% and 24% or between 6% and 23% or between 7% and 22% or between 8% and 20% of the polymer weight which exists on the antibody-polymer conjugate.

Any monoclonal antibody can be used as antibody in antibody-polymer conjugates prepared within the scope of the present invention. In a preferred embodiment of the present invention, the monoclonal antibody can be selected from a group comprising 3F8, 8H9, Abagovomab, Abciximab, Abituzumab, Abrezekimab, Abrilumab, Actoxumab, Adalimumab, Adecatumumab, Atidortoxumab, Aducanumab, Afasevikumab, Afelimomab, Alacizumab pegol, Alemtuzumab, Alirocumab, Altumomab pentetate, Amatuximab, Anatumomab mafenatox, Andecaliximab, Anetumab ravtansine, Anifrolumab, Anrukinzumab (IMA-638), Apolizumab, Aprutumab ixadotin, Arcitumomab, Ascrinvacumab, Aselizumab, Atezolizumab, Atinumab, Atorolimumab, Avelumab, Azintuxizumab vedotin, Bapineuzumab, Basiliximab, Bavituximab, bCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, CBR96-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, lanalumab, Ibalizumab, 1B1308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Llarcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab amadotin, Lutikizumab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140,Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab emtansine, TRBSO7, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab Zolbetuximab (=IMAB362, Claudiximab), Zolimomab aritox. In a preferred embodiment of the present invention, an antibody selected from a group comprising nivolulumab, pembrolizumab trastuzumab, cetuximab is used. In a particularly preferred embodiment of the present invention, trastuzumab is used.

The linker (L₂) used in the antibody-polymer conjugates according to the invention can be selected from cleavable linkers (L₁) or non-cleavable linkers as also mentioned in the definition given above. Said non-cleavable linkers describe the bonds which protect their forms under the physiologic conditions of the body and which do not fragment or which fragment in negligible amounts. The non-cleavable linker can be formed by C₁-C₁₀ hydrocarbon chain and optionally one or more carbons, which exist(s) in said hydrocarbon chain can be substituted with —O, —NH, —N, —S, —C(O) or with the combinations thereof and said carbons can be adjacent to or separate from each other. In a preferred embodiment of the present invention, linkers according to the invention can comprise one or more substitution like —COOH, —OH, —NH₂, —SH— in addition to —O, —NH, —N, —S, —C(O) substitutions or in an independent manner from —O, —NH, —N, —S, —C(O) substitutions. In a particularly preferred embodiment of the present invention, as the linker (L₂), —C(O)CH₂CH₂C(CH₃)(CN)— is used, wherein the carbonyl group shown by —C(O) reacts with —NH₂ provided on any amino acid provided in monoclonal antibody structure and is connected to the amino acid by means of amide bond.

The cleavable linker (L₁) used in the subject matter antibody-polymer conjugates can be any substance like poly(ethylene glycol), an amino acid, poly(amino acid) (for instance, peptide or oligo-peptide) or poly(peptide). In a preferred embodiment of the present invention, the cleavable linker can be selected from short peptides which have specific peptide sequences such as Gly-Phe-Leu-Gly which can be described as GFLG or Val-Cit or Phe-Lys or Val-Ala or Ala-Leu-Ala-Leu which can be broken by catepcine B.

The cleavable linker (L₁) used in the subject matter antibody-polymer conjugates can be C₁-C₁₀ hydrocarbon which is substituted with at least one acetal, ester, imine, amide, disulphide, carbamate or with binary or ternary combinations thereof.

In another embodiment of the present invention, the cleavable linker (L₁) can be the combination of a peptide chain selected from a group comprising Phe-Lys or Val-Ala or Ala-Leu-Ala-Leu or Val-Cit, GFLG and a substituted C₁-C₁₀ hydrocarbon comprising at least one functional group selected from a group comprising acetal, ester, imine, amide, disulphide, carbonate, carbamate, hydrazone.

In the case, where L2 is selected from cleavable linkers (L1), the L1 binding the drug moiety to the polymer backbone and L2 which is binding the polymer drug conjugate to the antibody are independent from each other and can be the same or different from another as can be seen in the below embodiments of the invention.

In a preferred embodiment of the present invention, the subject matter antibody-polymer conjugate can comprise one or a number of the below mentioned,
GFLG as the cleavable linker (L₁), and —COCH₂CH₂C(CH₃)(CN)— as the linker (L₂), an agent selected from the group of docetaxel, pemetrexed, hydroxychloroquine, combretastatin, gemcitabine, doxorubicin, 5-FU, 5′-Deoxy 5-Fluorocytidine, emtansine (5′-DFCR) and lapatinib as the therapeutic agent, and trastuzumab as the antibody.
Val-Cit as the cleavable linker (L₁), —COCH₂CH₂C(CH₃)(CN)— as the linker (L₂), an agent selected from the group of docetaxel, pemetrexed, hydroxychloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Phe-Lys as the cleavable linker (L₁), —COCH₂CH₂C(CH₃)(CN)— as the linker (L₂), an agent selected from the group of docetaxel, pemetrexed, hydroxychloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Val-Ala as the cleavable linker (L₁), —COCH₂CH₂C(CH₃)(CN)— as the linker (L₂), an agent selected from the group of docetaxel, pemetrexed, hydroxychloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Ala-Leu-Ala-Leu as the cleavable linker (L₁), —COCH₂CH₂C(CH₃)(CN)— as the linker (L₂), an agent selected from the group of docetaxel, pemetrexed, hydroxychloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Disulphide substituted C₁-C₁₀ hydrocarbon as the cleavable linker (L₁), —COCH₂CH₂C(CH₃)(CN)— as the linker (L₂), an agent selected from the group of docetaxel, pemetrexed, hydroxychloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Hydrazone substituted C₁-C₁₀ hydrocarbon as the cleavable linker (L₁), —COCH₂CH₂C(CH₃)(CN)— as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
GFLG as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Val-Cit as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Phe-Lys as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Val-Ala as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Ala-Leu-Ala-Leu as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Disulphide substituted C₁-C₁₀ hydrocarbon as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Hydrazone substituted C₁-C₁₀ hydrocarbon as the cleavable linker (L₁), disulphide substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
GFLG as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Val-Cit as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Phe-Lys as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Val-Ala as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Ala-Leu-Ala-Leu as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Disulphide substituted C₁-C₁₀ hydrocarbon as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.
Hydrazone substituted C1-C10 hydrocarbon as the cleavable linker (L₁), hydrazone substituted C₁-C₁₀ hydrocarbon as the linker (L₂), an agent selected from the group of docetaxel, pemetrexed, chloroquine, combretastatin, gemcitabine, doxorubicin, 5-FU, 5′-Deoxy 5-Fluorocytidine (5′-DFCR), emtansine and lapatinib as the therapeutic agent, and trastuzumab as the antibody.

In another aspect, a preferred embodiment of the present invention relates to a method (Method 1) for use in preparing antibody-polymer conjugates shown by Formula 1 wherein said method comprises the following steps:
a. obtaining RAFT agent comprising disulphide by reacting a trithiocarbonate or a dithioate molecule (RAFT agent), having carboxylic acid functional group thereon, with a C₁-C₁₀2-(pyridine-2-yl disulphanyl) alcohol,
b. obtaining the polymer-drug conjugate having disulphide group at one end thereof by reacting the RAFT agent, obtained in step a), with the (meth)acrylate monomer shown by Formula II

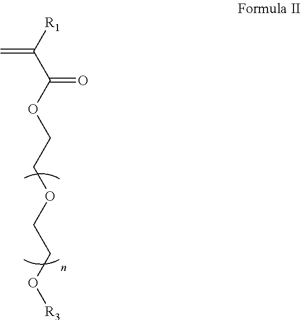

and (meth)acrylate-L₁-D monomer shown by Formula III

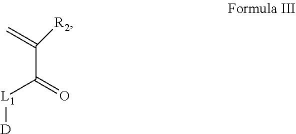

wherein R₁, R₂, R₃ are independently selected from —H or —CH₃, L₁ is a cleavable bond, D is a therapeutic agent and n is a natural number between 1 and 200,
c. treating the antibody with Traut's reagent (2-iminothiolane hydrochloride),
d. obtaining antibody-polymer conjugates of the invention (Formula 1) by reacting the polymer-drug conjugates obtained in step b) and the antibody obtained in step c) at pH between 6.0 and 7.5 in an aqueous solution and an organic solution.

In another aspect in a preferred embodiment of the present invention, the present invention relates to a method (Method 2) for use in preparing antibody-polymer conjugates shown by Formula 1, wherein said method comprises the following steps:
a. obtaining modified RAFT agent by reacting a trithiocarbonate or dithioate molecule (RAFT agent), having carboxylic acid functional group thereon, with N-hydroxy succinimide,
b. obtaining the polymer-drug conjugate having disulphide group at one end thereof by reacting the RAFT agent, obtained in step a), with the (meth)acrylate monomer shown by Formula II

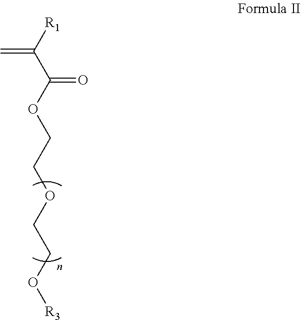

and (meth)acrylate-L₁-D monomer shown by Formula III,

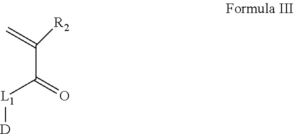

wherein R₁, R₂, R₃ are independently selected from —H or —CH₃, L₁ is a cleavable bond, D is a therapeutic agent and n is a natural number between 1 and 200,
c. obtaining the antibody-polymer conjugates (Formula 1) of the invention by reacting the polymer-drug conjugate obtained in step b) and the antibody obtained in step c) at pH between 6.0 and 7.5 in an aqueous solution and in an organic solution.

In method 1 and method 2, in step a) another functional group like —CN, —N₃ is optionally provided on trithiocarbonate or dithioate molecule where carboxylic acid functional group is provided thereon. Said trithiocarbonate or dithioate molecule, whereon carboxylic acid functional group is provided, can have any structure provided that there is a trithiocarbonate or dithioate group and at least one carboxylic acid group.

In method 1 and method 2, in the polymerization realized in step b), a second polymerization initiator can be used which is present in the known state of the art. In a preferred embodiment of the present invention, as the second polymerization initiator, 2,2′-Azobis(2-methyl-propionitril) (AIBN) or derivatives, for instance, 4,4′-Azobis(4-cyano-valeric acid), 4,4′-Azobis(4-cyano-valeric acid), 1,1′-Azobis(cyclo-hexane-carbo-nitrile), 2,2′-Azobis(2-metil-propionamidine) dihydro-chloride can be used.

In method 1 and method 2, the polymerization described in step b) is realized at a temperature between 25° C. and 100° C. and preferably at a temperature between 60° C. and 90° C.

In method 1 and method 2, the antibody-polymer conjugation is realized at a temperature between 20° C. and 45° C. and preferably at a temperature of room temperature, particularly preferably at a temperature of 25° C.

In an embodiment of the present invention, PEG (meth)acrylate (Formula II) is used which has an average molecular weight between 200 and 2000 grams/mole. In a preferred embodiment, PEG (meth)acrylate (Formula II) has an average molecular weight between 250 and 1500 grams/mole and in a more preferred embodiment, PEG (meth)acrylate (Formula II) has an average molecular weight between 300 and 1100 grams/mole. The subject matter PEG (meth)acrylate (Formula II) can have an average molecular weight of for instance 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000 grams/mole.

Monoclonal antibodies provided in antibody-polymer conjugates provide entry of the attached therapeutic agents into the cell when they go to the target region with the help of their affinity towards the highly expressed receptors in the cells. Thus, the targeted cell type changes according to the used mAb type. The subject matter antibody-polymer conjugates can be used in the treatment of different diseases according to the characteristics of the monoclonal antibody which forms the conjugate.

As used herein, the terms of “treatment” or “treating” describes elimination or reduction of at least one of biological markers (for instance, tumor or tumor markers) which show the presence of a disease or prevention, reduction, mitigation, healing or blocking of at least one symptom which characterizes a pathological disease in a subject where a disease diagnosis has been realized or who is determined to have high probability of having a disease.

The subject matter antibody-polymer conjugates in general can be applied in the form of a pharmaceutical composition.

Therefore, an embodiment of the present invention is pharmaceutical compositions comprising antibody-polymer conjugates shown by formula I and at least one pharmaceutically acceptable excipient.

The composition comprising the subject matter antibody-polymer conjugate can be at any suitable form based on the preferred method in terms of application of this composition to a patient. The composition comprising the subject matter antibody-polymer conjugates can be formulated in a manner applied orally for instance in the form of liquid dispersions or aqueous or oily suspensions or the composition can be formulated for parenteral application like for instance, subcutaneous, intravenous, intramuscular, intrasternal, intra-peritoneal, intradermal, transdermal or other infusion methods. The subject matter composition comprising polymer drug conjugates can also be formulated for application by means of respiratory route in the form of spray tube or for application as solution for application by means of respiratory device or nebulizer. The subject matter antibody-polymer conjugate is preferably applied transdermally, subcutaneously, intranasally, intravenously, intramuscularly, intratumorally. In any case, the most suitable form in terms of application will depend on the specific therapeutic agent provided in the subject matter antibody-polymer conjugate, the kind of the disease and the physical condition of the subject.

The subject matter antibody-polymer conjugates can be applied together for instance, simultaneously, sequentially or separately with one or more therapeutic agent which can be in the form of a cancer preventing substance or immune system regulating substance, antiviral, anti-infective, anti-microbial or anaesthetic substance or the combinations thereof.

Said second therapeutic agent can be any therapeutic agent provided that it is different from the substance which is present in the antibody-polymer conjugate of the invention.

Within the scope of this specification, it is aimed that the term comprising describes the term including.

In places where it is technically suitable, the embodiments of the invention can be combined.

The embodiments are explained here so as to comprise specific characteristics/elements. The description moreover covers the other embodiments comprising said characteristics/elements or formed by these.

Patents, applications, scientific publications and similar technical references have been included in this document by means of reference.

The embodiments which are described here in a specific manner and clearly described can form the base of a disclaimer in a stand-alone manner together with one or a number of other embodiments.

Now the present invention will be explained with reference to the examples given below which are only for exemplary purposes and which shall not be interpreted as delimiting the scope of the present invention in any manner.

### EXAMPLES

### Example 1: The Synthesis Method for 2-(pyridin-2-yl-disulfanyl) ethyl 4-cyano-4-(((dodecylthio) carbon-thioyl) thio) pentanoate (PDS-CDTPA)

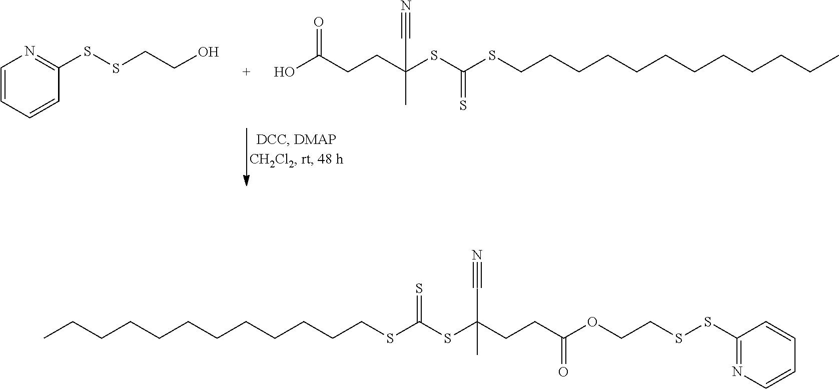

2-(pyridine-2-yl sulphonyl) ethanol (41.7 mg, 0.223 mmol) is dissolved in 4-cyano-4-(dodesyl sulphanyl thio-carbonyl) sulphanyl pentanoic acid (60 mg, 0.148 mmol) and 4-dimethyl-amino-pyridine (DMAP) (18 mg, 0.147 mmol) CH₂Cl₂ (2 mL). The reaction mixture is cooled to 0° C. in an ice bath, and N,N-dicyclohexyl-carbodiimide (DCC) (36.8 mg, 0.178 mmol) dissolved in CH₂Cl₂ (2 mL) is added to the reaction mixture dropwise. The reaction is stirred at 25° C. The formed dicyclohexyl urea (DCU) is filtered and removed. ¹H NMR (CDCl₃, δ, ppm) 8.49 (d, 1H, J=3.8 Hz), 7.67-7.64 (m, 2H), 7.12 (bs, 1H), 4.38 (t, 2H, J=6.3 Hz), 3.33 (t, 2H, J=7.4 Hz), 3.05 (t, 2H, J=6.3 Hz), 2.63-2.33 (m, 4H), 1.87 (s, 3H), 1.68 (dd, 2H, J=14.5, 7.1), 1.39-1.26 (m, 18H), 0.88 (t, 3H, J=6.4 Hz).

### Example 2: The Synthesis Method for 4-Cyano-4-(phenyl-carbono-thioyl-thio) Pentanoic Succinimide Ester (SCPDB)

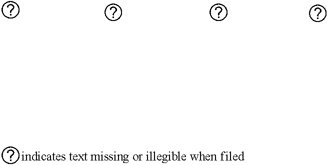

4-cyano-4-(phenyl-carbono-thioyl-thio) pentanoic acid (CPADB) (200 mg, 0.72 mmol) is dissolved in N-hydroxy succinimide (NHS) (91 mg, 0.79 mmol) CH₂Cl₂ (1.5 mL). N,N-Dicyclohexylcarbodimide (DCC) (163 mg, 0.79 mmol) is dissolved in CH₂Cl₂ (0.5 mL) and is added dropwise to the reaction mixture in the ice bath. The reaction is stirred for 3 hours at room temperature. The obtained raw product is precipitated in cold ethyl acetate and purified by means of product column chromatography. ¹H NMR (400 MHz, CDCl₃, δ, ppm) δ 7.93 (d, J=7.4 Hz, 2H), 7.58 (t, J=7.4 Hz, 1H), 7.41 (t, J=7.8 Hz, 2H), 3.03-2.95 (m, 2H), 2.86 (s, 4H), 2.79-2.69 (m, 1H), 2.61-2.52 (m, 1H), 1.96 (s, 3H).

### Example 3: Synthesis of PDS-POEGMEMA-DTX Copolymer

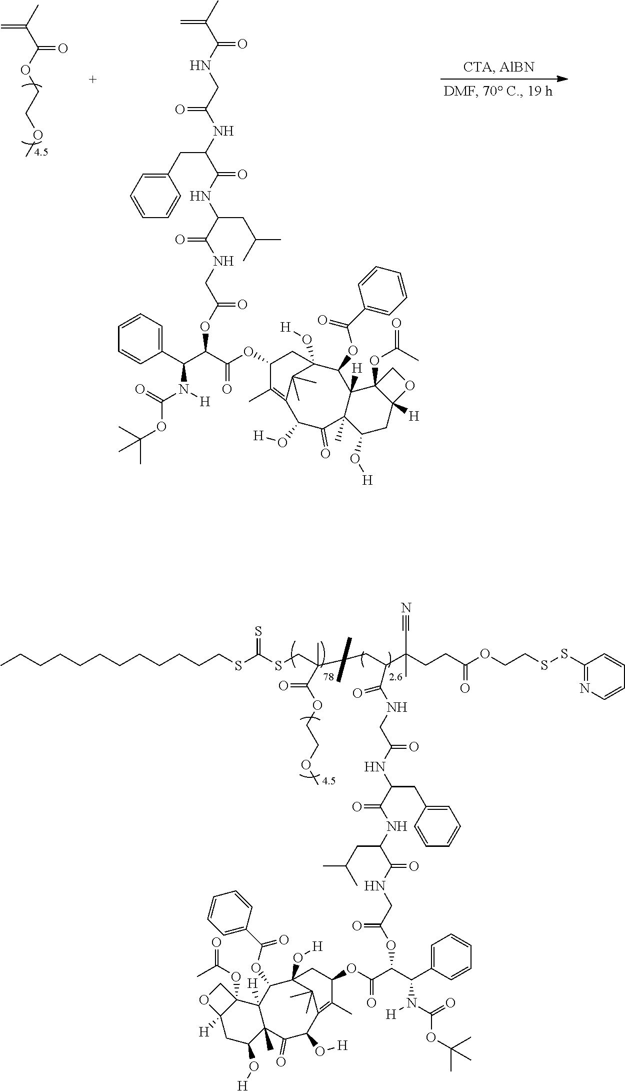

Polyethylene glycol monomethyl methacrylate (PEGMEMA) (100 mg, 0.33 mmol) is dissolved in DMF (0.3 mL). PDS-CDTPA (6.8 mg, 0.01 mmol) is dissolved in DMF (0.1 mL) and AIBN (0.39 mg, 2.4×10⁻³ mmol) is dissolved in DMF (0.1 mL). The prepared solutions are added into PEGMEMA solution under N₂ atmosphere. MA-GFLG-DTX (45 mg, 0.04 mmol) solution, prepared by dissolving in DMF (0.4 mL) beforehand is added to the reaction mixture. The reaction is stirred at temperature of 80° C. DMF is removed after the reaction.

The obtained product is precipitated in ether, and the formed precipitate is removed by filtration and purified by means of dialysis membrane. The molecular weight of the obtained polymer is detected by means of GPC (10 kDa). From the ¹H NMR result, it has been detected that there is drug with proportion of 10.6% by weight in the polymer chain.

### Example 4: NHS-POEGMEMA-DTX Copolymer Synthesis Method

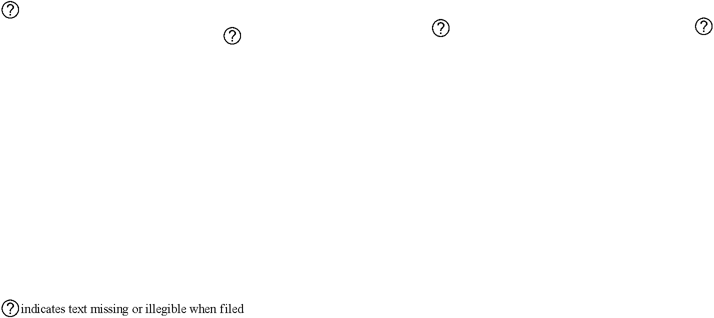

PEGMEMA (86 mg, 0.29 mmol), MA-GFLG-DTX (120 mg, 0.096 mmol) and SCPDB (3.6 mg, 0.096 mmol) are dissolved in DMF (1.00 mL). AIBN (0.36 mg, 0.0019 mmol) is added into the reaction mixture. DMF is removed after polymerization. The obtained raw product is purified by means of dialysis. The molecular weight of the obtained polymer is detected by means of GPC (10 kDa). From the ¹H NMR result, it has been detected that there is drug with proportion of 12% by weight in the polymer chain.

### Example 5: Trastuzumab Thiol (Tmab-SH) Preparation Method

2-Imino-thiolane (0.1 mg, 3.6×10⁻³ mmol) is dissolved in PBS (3 mL, pH 8.2). Trastuzumab (Tmab) (4.6 mg/mL, 3.1×10−5 mmol) is added to the reaction. The reaction is stirred for 2.5 hours at 25° C. The number of thiol groups attached to Tmab is determined by means of Ellman procedure (1). As a result of this experiment, it has been seen that 6 thiols are obtained on each Tmab. Dialysis is made with PBS buffer solution (pH 6.5). In order to determine Tmab concentration, BCA analysis is made (2). As a result of the analysis, it has been detected that Tmab concentration is 3.7 mg/mL.

### Example 6: PDS-POEGMEMA-DTX Copolymer and Tmab-SH Conjugation

Tmab-SH and PDS-POEGMEMA-DTX Copolymer is dissolved in DMSO and PBS buffer solution (pH 6.5). The obtained mixture is stirred at room temperature. The obtained product is purified by means of conventional methods.

### Example 7: NHS-POEGMEMA-DTX Copolymer and Tmab Conjugation

Tmab and NHS-POEGMEMA-DTX copolymer is dissolved in DMSO and PBS buffer solution (pH 6.5). The obtained mixture is stirred at room temperature. The obtained product is purified by means of conventional methods.

### REFERENCES

1. “DTNB (Ellman's Reagent) (5,5-Dithio-Bis-(2-Nitrobenzoic Acid)—Thermo Fisher Scientific,” 16 Jul. 2019.
2. Associate, A., “User Guide: Pierce BCA Protein Assay Kit (MAN0011430 Rev. A).” 16 Jul. 2019.

1.-28. (canceled)

29. An antibody-polymer conjugate comprising 1.1 to 13 polymer-drug conjugates which have been conjugated with cleavable or non-cleavable bonds to a monoclonal antibody, wherein the polymer which carries therapeutic agent comprises (meth)acrylate backbone and has poly(ethylene glycol) at the side branches thereof and the therapeutic agent is connected to the polymer backbone by means of a cleavable bond.

30. The antibody-polymer conjugate according to claim 29, wherein said conjugate has the structure shown by Formula I; 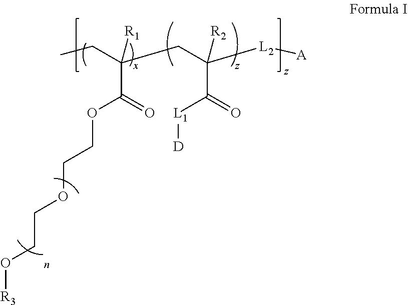
wherein;
R₁, R₂, R₃ are selected from —H or —CH₃ in an independent manner from each other,
x is a natural number between 1 and 140,
y is a natural number between 1 and 40,
z is a natural number between 1.1 and 13,
L₁ is a cleavable linker,
L₂ is a linker,
D is a therapeutic agent,
A is a monoclonal antibody.

31. The antibody-polymer conjugate according to claim 30, wherein it is random copolymer or block copolymer.

32. The antibody-polymer conjugate according to claim 29, wherein the therapeutic agent is selected from a group comprising anti-neoplastic agents, immune system modulating agents, agents which show anti-angiogenic characteristic on malign cells, agents which inhibit cell proliferation on malign cells or agents which increase immune cell proliferation and wherein the anti-neoplastic agent is selected from a group comprising nucleoside analogs, antifolates, other metabolites, topoisomerase I inhibitors, anthracyclins, podophyllotoxins, taxanes, vinca alkaloids, alkylating substances, platin compounds, anti-hormones, radio-pharmaceuticals, tyrosine kinase inhibitors, rapamycin protein complex mammalian target (mTOR) inhibitors, retinoids, immune system regulating substances, histonedeacetylase inhibitors and other substances.

33. The antibody-polymer conjugate according to claim 29, wherein the monoclonal antibody is selected from a group comprising 3F8, 8H9, Abagovomab, Abciximab, Abituzumab, Abrezekimab, Abrilumab, Actoxumab, Adalimumab, Adecatumumab, Atidortoxumab, Aducanumab, Afasevikumab, Afelimomab, Alacizumab pegol, Alemtuzumab, Alirocumab, Altumomab pentetate, Amatuximab, Anatumomab mafenatox, Andecaliximab, Anetumab ravtansine, Anifrolumab, Anrukinzumab (IMA-638), Apolizumab, Aprutumab ixadotin, Arcitumomab, Ascrinvacumab, Aselizumab, Atezolizumab, Atinumab, Atorolimumab, Avelumab, Azintuxizumab vedotin, Bapineuzumab, Basiliximab, Bavituximab, bCD-100, Bectumomab, Begelomab, Belantamab mafodotin, Belimumab, Bemarituzumab, Benralizumab, Berlimatoxumab, Bermekimab, Bersanlimab, Bertilimumab, Besilesomab, Bevacizumab, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, CBR96-doxorubicin immunoconjugate, Cedelizumab, Cemiplimab, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, Crizanlizumab, Crotedumab, CR6261, Cusatuzumab, Dacetuzumab, Daclizumab, Dalotuzumab, Dapirolizumab pegol, Daratumumab, Dectrekumab, Demcizumab, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotuzumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emapalumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab, Exbivirumab, Fanolesomab, Faralimomab, Faricimab, Farletuzumab, Fasinumab, FBTA05, Felvizumab, Fezakinumab, Fibatuzumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, lanalumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lampalizumab, Lanadelumab, Landogrozumab, Laprituximab emtansine, Llarcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab amadotin, Lutikizumab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140,Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilotumumab, Rinucumab, Risankizumab, Rituximab, Rivabazumab pegol, Robatumumab, Rmab, Roledumab, Romilkimab, Romosozumab, Rontalizumab, Rosmantuzumab, Rovalpituzumab tesirine, Rovelizumab, Rozanolixizumab, Ruplizumab, SA237, Sacituzumab govitecan, Samalizumab, Samrotamab vedotin, Sarilumab, Satralizumab, Satumomab pendetide, Secukinumab, Selicrelumab, Seribantumab, Setoxaximab, Setrusumab, Sevirumab, Sibrotuzumab, SGN-CD19A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Tildrakizumab, Tigatuzumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab emtansine, TRBSO7, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Vunakizumab, Xentuzumab, XMAB-5574, Zalutumumab, Zanolimumab, Zatuximab, Zenocutuzumab, Ziralimumab Zolbetuximab (=IMAB362, Claudiximab), Zolimomab aritox preferably nivolulumab, pembrolizumab trastuzumab, cetuximab, most preferably trastuzumab.

34. The conjugate according to claim 29, wherein the linker (L₂) is a cleavable or a non-cleavable linker.

35. The conjugate according to claim 34, wherein the non-cleavable linker comprises the C₁-C₁₀ hydrocarbon chain, preferably one or more carbon, provided in C₁-C₁₀ hydrocarbon chain, is independently substituted with —O, —NH, —N, —S, —C(O) or the combination thereof.

36. The conjugate according to claim 35, wherein C₁-C₁₀ hydrocarbon chain can comprise one or more substituent like —CN, —COOH, —OH, —NH₂ and —SH.

37. The conjugate according to claim 29, wherein the cleavable linker (L₁) is a C₁-C₁₀ hydrocarbon comprising at least one functional group selected from a group comprising acetal, ester, imine, amid, disülfit, carbonate, carbamate, hydrazone or a peptide chain selected from a group comprising GFLG, Val-Cit, Phe-Lys, Val-Ala, Ala-Leu-Ala-Leu.

38. A method (Method 1) for usage in preparation of antibody-polymer conjugates according to claim 29, wherein said method comprises the steps of:
a. obtaining RAFT agent comprising disulphide by reacting a trithiocarbonate or dithioate molecule (RAFT agent), having carboxylic acid functional group thereon, with a C₁-C₁₀2-(pyridine-2-yl disulphanyl) alcohol,
b. obtaining the polymer with drug having disulphide group at one end thereof by reacting the RAFT agent, obtained in step a), with the (meth)acrylate monomer shown by Formula II 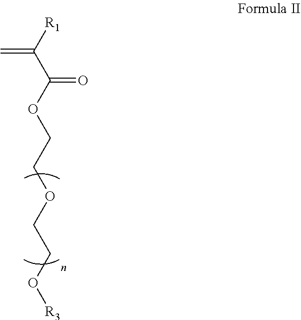
and (meth)acrylate-L₁-D monomer shown by Formula III 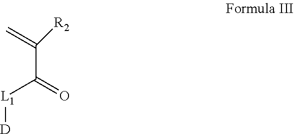
wherein R₁, R₂, R₃ are independently selected from —H or —CH₃, L₁ is a cleavable linker, D is a therapeutic agent and n is a natural number between 1 and 200,
a. treating the antibody with Traut's reagent (2-imino-thiolane hydro-chloride),
b. obtaining the antibody-polymer conjugate according to invention (Formula 1) by reacting the polymer with drug obtained in step b) and the antibody obtained in step c) at pH between 6.0 and 7.5 in an aqueous solution with an organic solution.

39. A method (Method 2) for use in preparation of antibody-polymer conjugates according to claim 29, wherein the said method comprises the steps of:
a. obtaining modified RAFT agent by reacting a trithiocarbonate or dithioate molecule (RAFT agent), having carboxylic acid functional group thereon, with N-hydroxy succinimide,
b. obtaining the polymer with drug having disulphide group at one end thereof by reacting the RAFT agent, obtained in step a), with the (meth)acrylate monomer shown by Formula II 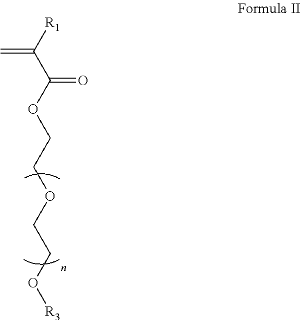
and (meth)acrylate-L₁-D monomer shown by Formula III, 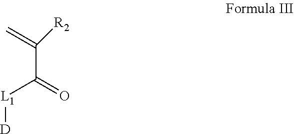
wherein R₁, R₂, R₃ are independently selected from —H or —CH₃, L₁ is a cleavable linker, D is a therapeutic agent and n is a natural number between 1 and 200,
c. obtaining the subject matter antibody-polymer conjugates (Formula 1) by reacting the polymer with drug obtained in step b) and the antibody obtained in step c) at pH between 6.0 and 7.5 in and aqueous solution with an organic solution.

40. Pharmaceutical compositions comprising antibody-polymer conjugates according to claim 29 as the active substance.

41. The pharmaceutical composition according to claim 40, wherein said composition comprises a second therapeutic agent preferably selected from a group comprising anti-neoplastic agents, antiviral, anti-infective, anti-microbial or anaesthetic substances provided that it is different from the therapeutic agent provided on the antibody-polymer conjugate.
